(19) United States
(12) Patent Application Publication
(10) Pub. No.: US 2023/0157993 A1
(43) Pub. Date: May 25, 2023

(54) PHARMACEUTICAL COMPOSITION FOR TREATING OR PREVENTING ALOPECIA INCLUDING DECURSIN

(71) Applicant: University-Industry Cooperation Group of Kyung Hee University, Yongin-si (KR)

(72) Inventors: Woong Mo YANG, Hanam-si (KR); Mi Hye KIM, Suwon-si (KR)

(73) Assignee: University-Industry Cooperation Group of Kyung Hee University, Yongin-si (KR)

(21) Appl. No.: 17/985,174

(22) Filed: Nov. 11, 2022

(30) Foreign Application Priority Data
Nov. 24, 2021 (KR) 10-2021-0163325

(51) Int. Cl. A61K 31/366; A61P 17/14; A61Q 7/00; A61K 8/49; A61K 8/9789; A61K 36/232; A61K 9/16; A61K 9/48; A61K 9/00; A61Q 5/02; A23L 33/105

(52) U.S. Cl. CPC A61K 31/366; A61P 17/14; A61Q 7/00; A61K 8/498; A61K 8/9789; A61K 36/232; A61K 9/1617; A61K 9/1623; A61K 9/1652; A61K 9/4858; A61K 9/4866; A61K 9/4825; A61K 9/0095; A61Q 5/02; A23L 33/105; A23V 2002/00

(57) ABSTRACT

The present disclosure relates to a composition for treating or preventing alopecia including decursin, wherein when administered externally or orally, the decursin promotes secretion of KGF in hair follicle tissues, prevents apoptosis of hair follicle cells, and exhibits the effect of promoting hair growth, thereby being useful for the treatment or prevention of alopecia.

### CROSS-REFERENCE TO RELATED APPLICATION

This application claims the benefit of Korean Patent Application No. 10-2021-0163325 filed on Nov. 24, 2021, in the Korean Intellectual Property Office, the entire disclosure of which is incorporated herein by reference for all purposes.

### BACKGROUND

### 1. Field of the Invention

The present disclosure relates to a composition for treating or preventing alopecia, including decursin that promotes expression of keratinocyte growth factor (KGF) in skin tissues.

### 2. Description of the Related Art

Alopecia is a hairless condition on a spot where there should normally be hair, of which the main cause is known to be genetic factors. Recently, along with the increase in social stress as well as environmental pollution, westernized eating habits such as instant food, frequent perming and hair dyeing, and incorrect scalp care, the number of people with alopecia is gradually increasing. However, the exact cause of alopecia has not been identified yet, people who are suffering therefrom have been increasing, and age group is becoming younger.

As a conventional treatment method for alopecia, there have been preparations mainly based on female hormones in relation to hormone theory. However, due to reports of skin inflammation and side effects caused by hormone administration, use of the preparations has been discontinued. Typical hair growth agents that have been recently used are minoxidil (6-amino-1,2-dihydro-1-hydroxy-2-imino-4-phenoxypyrimidine) disclosed in U.S. Pat. No. 5,215,894 and finasteride from Merck stated in U.S. Pat. No. 5,215,894, which were developed to be used for the purpose of promoting blood circulation at first but then known to derive a hair growth effect as a side effect among patients using the same. Since then, they were approved by the U.S. Food and Drug Administration (FDA) as a raw material for hair growth and used as hair growth treatment.

However, in the case of minoxidil, a sticky feeling of use and side effects causing skin irritation have been reported. In the case of finasteride which is currently used as a formulation for oral administration, side effects such as sexual dysfunction have been reported due to administration. In addition, there is a disadvantage that it is effective for alopecia only when regular administration is followed.

Therefore, the demand for new drugs for ameliorating or treating alopecia that may reduce the side effects of existing drugs is gradually increasing.

### PRIOR ART DOCUMENT

### Patent Document

Korean Patent Application Publication No. 10-2020-0033224 (Published on Mar. 27, 2020)

### SUMMARY

### Problem to be Solved by the Invention

An object of the present disclosure is to provide a pharmaceutical composition, a health functional food composition, and a cosmetic composition for preventing or treating alopecia through discovery of a novel material capable of preventing or treating alopecia.

### Means for Solving the Problem

The present disclosure provides a pharmaceutical composition for treating or preventing alopecia, including decursin or a pharmaceutically acceptable salt thereof.

In addition, the present disclosure provides a pharmaceutical composition for treating or preventing alopecia in which expression of keratinocyte growth factor (KGF) in the skin tissue is suppressed, including decursin or a pharmaceutically acceptable salt thereof.

In addition, the present disclosure provides a health functional food composition for preventing or ameliorating alopecia including decursin.

In addition, the present disclosure provides a cosmetic composition for preventing or ameliorating alopecia including decursin.

### Effects of the Invention

According to the present disclosure, when administered externally or orally, decursin promotes secretion of KGF in hair follicle tissues, prevents apoptosis of hair follicle cells, and exhibits the effect of promoting hair growth, thereby being useful for treatment or prevention of alopecia.

### BRIEF DESCRIPTION OF THE DRAWINGS

FIG. 1 shows an analysis of an effect of decursin on hair growth in mice with alopecia by morphological changes (magnification ×10) of hair growth photographed with a dermatoscope.

FIG. 2 shows an analysis of an effect of decursin on hair growth in mice with alopecia by histological findings (magnification ×100) of dorsal skin tissues stained with hematoxylin and eosin.

FIG. 3 shows an analysis of an effect of decursin on KGF expression in mice with alopecia using immunofluorescence images (green fluorescence intensity indicates KGF, magnification is ×400) of KGF⁺ expression in hair follicles of dorsal skin tissues.

FIG. 4 shows an analysis of an effect of decursin on KGF expression in mice with alopecia on the basis of a level of protein expression of KGF in skin tissues.

FIG. 5 shows an analysis of an effect of decursin on apoptosis-related factors in keratinocytes.

### DETAILED DESCRIPTION

Hereinafter, the present disclosure will be described in detail.

The inventors completed the present disclosure by first discovering the effect of promoting KGF secretion in hair follicle tissues, preventing apoptosis of hair follicle cells, and promoting hair growth when externally or orally administrating decursin which is known to have various biological activities including anti-inflammatory, anti-cancer, and antioxidant effects.

Accordingly, the present disclosure provides a pharmaceutical composition for treating or preventing alopecia, including decursin or a pharmaceutically acceptable salt thereof.

The decursin is a compound represented by the following Chemical Formula 1. The decursin may be extracted from the root of Angelica gigas, but may be prepared by various methods such as chemical synthesis:

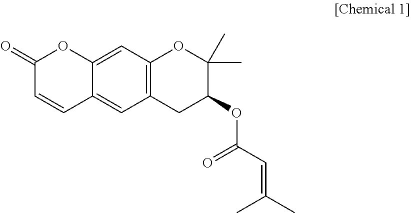 [SMILES: CC(C)=CC(=O)O[C@H]1Cc2cc3ccc(=O)oc3cc2OC1(C)C]

The decursin may promote the growth of hair fibers inhibited in hair follicle tissues. In particular, the decursin may promote expression of keratinocyte growth factor (KGF) in the skin tissue, prevent apoptosis of hair follicle cells, and promote hair growth.

The alopecia may be one or more selected from the group consisting of androgenetic alopecia, telogen alopecia, drug-induced alopecia, mechanical alopecia, traumatic alopecia, pressure-induced alopecia, anagen alopecia, dandruff-induced alopecia, syphilitic alopecia, seborrheic alopecia, symptomatic alopecia, scarring alopecia, congenital alopecia, alopecia areata, tinea capitis, frontal alopecia, hypotrichosis, hereditary hypotrichosis simplex, and alopecia universalis, but is not limited thereto.

The drug-induced alopecia may be alopecia induced by chemotherapy which is a treatment that involves administration of an anticancer agent to destroy cancer cells. Anticancer drugs that attack cancer cells and interfere with the growth thereof also affect normal cells, and pore cells are also affected by anticancer drugs, which may lead to alopecia. The chemotherapy-induced alopecia may be induced by an anticancer drug selected among cyclophosphamide, docetaxel, etoposide, ifosfamide, or paclitaxel, but is not limited thereto.

In addition, the present disclosure provides a pharmaceutical composition for treating or preventing alopecia in which expression of keratinocyte growth factor (KGF) in skin tissues is suppressed, including decursin or a pharmaceutically acceptable salt thereof.

The ‘pharmaceutically acceptable salt’ may be used in a form of any one of a pharmaceutically acceptable basic salt or acidic salt. The basic salt may be used in the form of any one of an organic base salt and an inorganic base salt such as a sodium salt, a potassium salt, a calcium salt, a lithium salt, a magnesium salt, a cesium salt and an aminium salt, an ammonium salt, a triethylamine salt, and a pyridinium salt, but is not limited thereto.

In addition, an acid added salt formed by free acid may be useful as the acid salt. As free acids, inorganic acids and organic acids may be used. As inorganic acids, hydrochloric acid, bromic acid, sulfuric acid, sulfurous acid, and phosphoric acid may be used. As organic acids, citric acid, acetic acid, maleic acid, fumaric acid, gluconic acid, methanesulfonic acid, benzenesulfonic acid, camphorsulfonic acid, oxalic acid, malonic acid, glutaric acid, acetic acid, glycolic acid, succinic acid, tartaric acid, 4-toluenesulfonic acid, galacturonic acid, embonic acid, glutamic acid, citric acid, and aspartic acid may be used. Preferably, hydrochloric acid may be used as the inorganic acid, and methanesulfonic acid as the organic acid.

In addition, the composition according to the present disclosure may all include salts, hydrates, and solvates that may be prepared by conventional methods as well as pharmaceutically acceptable salts.

The addition salt according to the present disclosure may be prepared by a conventional method. For example, preparation may be carried out by dissolving LGK974 in a water-miscible organic solvent, such as acetone, methanol, ethanol, or acetonitrile, and then adding an excess of an organic base or adding basic aqueous solution of an inorganic base for precipitation or crystallization. Alternatively, preparation may be carried out by evaporating the solvent or excess base from the mixture, and then drying the mixture to obtain an addition salt or suction-filtrating the precipitated salt.

According to an example embodiment of the present disclosure, in the skin treated with decursin, morphological hair growth and histological recovery of hair follicles were observed in mice with alopecia, and KGF+ fluorescence and protein expression were significantly increased by decursin treatment. In addition, it was also found that expression of caspase-3, -7, and -8 induced by TNF-α was decreased in a dose-dependent manner along with the suppression of expression of PI3K, AKT, ERK, and p38 in decursin-treated keratinocytes.

The pharmaceutical composition may be provided in one or more formulations selected from the group consisting of gels, emulsions, injections, powder, granules, aerosols, pastes, transdermal absorption agents, and patches according to a conventional method, but is not limited thereto.

In another example embodiment of the present disclosure, the pharmaceutical composition may further include one or more additives selected from the group consisting of carriers, excipients, disintegrants, sweeteners, coating agents, swelling agents, lubricants, polishing agents, flavoring agents, antioxidants, buffers, bacteriostatic agents, diluents, dispersants, surfactants, binders, and lubricants that are suitable and commonly used in the preparation of pharmaceutical compositions.

Specifically, as the carriers, excipients, and diluents, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil may be used. Solid preparations for oral administration may include tablets, pills, powder, granules, and capsules, and such solid preparations may be prepared by mixing at least one excipient, for example, starch, calcium carbonate, sucrose or lactose, and gelatin in the composition. In addition to simple excipients, lubricants such as magnesium stearate and talc may also be used. Liquid formulations for oral use may include suspensions, solutions, emulsions, and syrups, and various excipients, such as wetting agents, sweeteners, fragrances, and preservatives, may be included in addition to commonly used simple diluents such as water and liquid paraffin. Formulations for parenteral administration may include sterile aqueous solutions, non-aqueous solutions, suspensions, emulsions, freeze-dried preparations, and suppositories. Propylene glycol, polyethylene glycol, vegetable oils such as olive oil, and injectable esters such as ethyl oleate may be used as the non-aqueous solvents and suspensions. As a base material for the suppository, witepsol, macrogol, tween 61, cacao butter, laurin, and glycerogelatin may be used.

The pharmaceutical composition may be administered to a subject in a conventional manner via intravenous, intraarterial, intraperitoneal, intramuscular, intraarterial, intraperitoneal, intrasternal, transdermal, intranasal, inhalation, topical, rectal, oral, intraocular, or intradermal routes.

The preferred dosage of decursin may vary depending on the condition and weight of the subject, the type and extent of the disease, the drug form, the route and duration of administration, and may be appropriately selected by those skilled in the art. According to an example embodiment of the present disclosure, although not limited thereto, the daily dose may be 0.01 to 200 mg/kg, specifically 0.1 to 200 mg/kg, and more specifically 0.1 to 100 mg/kg. Administration may be conducted once a day or in several divided doses, but the scope of the present disclosure is not limited thereby.

In the present disclosure, the term ‘subject’ as used herein may refer to a mammal including human, but is not limited to the examples.

In addition, the present disclosure provides an external preparation for treating or preventing alopecia, including decursin or a pharmaceutically acceptable salt thereof.

The external preparation may be provided in a formulation selected from the group consisting of ointments, creams, gels, patches, sprays, plasters, lotions, liniment agents, pastas, and cataplasmas, but is not limited thereto.

In addition, the present disclosure provides a health functional food composition for preventing or ameliorating alopecia, including decursin.

The term “health functional food” as used herein refers to food manufactured and processed using raw materials or ingredients having useful functionality for the human body in accordance with the Law for Health Functional Foods, and “functionality” refers to intake for the purpose of obtaining useful effects for health purposes such as regulating nutrients for structures and functions of human body or physiological effects.

The health functional food composition of the present disclosure may include conventional food additives. Whether it is suitable as a “food additive” is determined by standards and criteria for the item in accordance with general rules and general test methods of Korean Food Additives Code approved by the Ministry of Food and Drug Safety, unless otherwise specified.

The items listed in the “Korean Food Additives Code” may include, for example, chemical compounds such as ketones, glycine, potassium citrate, nicotinic acid, and cinnamic acid, natural additives such as persimmon colors, licorice extracts, crystalline cellulose, sorghum colors, and guar gum, and mixed preparations such as sodium L-glutamate preparations, noodle-added alkali agents, preservative agents, and tar color agents.

The health functional food composition of the present disclosure may be manufactured and processed in the form of tablets, capsules, powder, granules, liquids, and pills. For example, of the health functional foods in the form of capsules, hard capsules may be prepared by mixing and filling the composition according to the present disclosure in a conventional hard capsule along with additives such as excipients. Soft capsules may be prepared by mixing the composition according to the present disclosure with additives such as excipients and filling the mixture in the capsule base such as gelatin. The soft capsules may include plasticizers such as glycerin or sorbitol, colorants, and preservatives, if necessary. The definitions of terms for the excipients, binders, disintegrants, lubricants, bitters, and flavoring agents are described in document known in the art, including ones with the same or similar functions. There is no particular limitation for the type of food, and health functional foods in the ordinary sense may all be included.

The term “prevention” as used herein refers to any action of suppressing or delaying a disease by administration of the composition according to the present disclosure. The term “treatment” as used herein refers to any action that improves or beneficially changes the symptoms of a disease by administration of the composition according to the present disclosure. The term “improvement” as used herein refers to any action that improves a bad condition of a disease by administering or ingesting the composition of the present disclosure to a subject.

In addition, the present disclosure provides a cosmetic composition for preventing or ameliorating alopecia, including decursin.

The cosmetic composition according to the present disclosure may be prepared in any formulation conventionally prepared in the art, and provided in the formulation selected from the group consisting of hair tonic, hair conditioner, hair essence, hair lotion, hair nutrition lotion, hair shampoo, hair conditioner, hair treatment, hair cream, hair nourishment cream, hair moisture cream, hair massage cream, hair wax, hair aerosol, hair pack, hair nourishment pack, hair soap, hair cleansing foam, hair oil, hair dryer, hair preservative, hair dye, hair wave agent, hair bleach, hair gel, hair glaze, hair dresser, hair lacquer, hair moisturizer, hair mousse, and hair spray, but is not limited thereto.

The cosmetic composition may include an acceptable carrier in the hair cosmetic preparation in addition to the decursin. Examples of the carrier may include alcohol, oil, surfactants, fatty acid, silicone oil, preservatives, humectants, moisturizers, viscosity modifiers, emulsions, stabilizers, sunscreens, color developers, fragrances, and diluents. Since specific compounds or compositions that may be used as the alcohol, oil, surfactants, fatty acid, silicone oil, preservatives, humectants, moisturizers, viscosity modifiers, emulsions, stabilizers, sunscreens, color developers, fragrances, and diluents are already known in the art, those skilled in the art may select and use the appropriate compounds or compositions.

In addition, the cosmetic composition may further include skin absorption accelerators, scalp protectors, or scalp activators in order to enhance the effect and minimize side effects.

In addition, the present disclosure provides a method for treating alopecia, including administering decursin or a pharmaceutically acceptable salt thereof.

In addition, the present disclosure provides a reagent composition for promoting expression of keratinocyte growth factor (KGF) in skin tissues, including decursin or a pharmaceutically acceptable salt thereof.

Hereinafter, the present disclosure will be described in detail through examples. These examples are merely for illustrating the present disclosure more specifically, and it is apparent to those of ordinary skill in the art to which the present disclosure pertains that the scope of the present disclosure is not limited by the examples according to the gist of the present disclosure.

### <Example 1> Examination of Preventive or Therapeutic Effects for Alopecia Using Decursin

1. Classification of Experimental Group and Animal Testing

All animal testing procedures were approved by the Laboratory Animal Care and Use Committee of Kyung Hee University (KHUASP(SE)-13-046). 5-week-old male C57BL/6J mice were purchased from Raonbio Co., Ltd. (Yongin, Korea). All mice were kept in plastic cages at 22±1° C. under 50±5% humidity with 12 hours of light-dark cycles. After 1 week of adaptation, mice were randomly assigned to 6 groups: (1) CTR, normal control group, (2) CYP, cyclophosphamide-induced alopecia group, (3) DEX, dexamethasone (DEX) treated in CYP-induced alopecia mice as positive controls, (4) D1, 1 μM decursin treated in CYP-induced alopecia mice, (5) D10, 10 μM decursin treated in CYP-induced alopecia mice, and (6) D100, 100 μM decursin treated in CYP-induced alopecia mice.

To synchronize the phases of the hair growth cycle, the dark hairs on the dorsal skin of all mice except the CTR group were shaved on day 0 using a shaving cream. 9 days later, 150 mg/kg of CYP (Sigma-Aldrich, MO, USA) was dissolved in saline and injected one time intraperitoneally into all mice except the CTR group. Injection of CYP induced the stage of hair follicles to turn into dystrophic catagen. From day 9 to day 16, 100 μL of 1, 10 and 100 μM decursin (Sigma-Aldrich) in saline containing 0.1% dimethyl sulfoxide was topically treated onto the shaved dorsal skin once a day. As a positive control, 0.1% DEX was administered topically to the shaved area on days 1, 3, 5, 7, and 9 to 16. Physiological saline was treated to the CTR and CYP groups during the experiment. In the beginning of the experiment on day 17, all mice were sacrificed under anesthesia.

2. Skin Monitoring Through a Digital Dermatoscope

The digital dermatoscope used in the present disclosure was a Smart Microscope Pro (Kangjin Technology, Seoul, Korea) in ×10 magnification. In all mice, dermoscopic images were acquired in the same area, that is, the central area of the dermal skin sample to which the treatment was applied.

3. Histological Analysis

The dorsal skin tissues of all sacrificed mice was collected and incubated in 10% neutralized formalin for 24 hours. After dehydration with ethanol and xylene, the dorsal skin tissues were embedded in paraffin. Skin specimens were sliced in 7 μm thickness and stained with hematoxylin and eosin (H&E) solutions.

4. Immunofluorescence Assay

Paraffin tissue sections were hydrated with xylene and ethanol, followed by blocking with 1% bovine serum albumin (BSA). The slides were incubated overnight at 4° C. with primary anti-rabbit keratinocyte growth factor (KGF) antibody. Secondary HRP-conjugated fluorescent antibody was treated onto tissue slides for 1 hour at room temperature. The stained skin tissues were observed with a fluorescence microscope (LSM 5 PASCAL; Carl Zeiss, Oberkochen, Germany).

5. Cell Treatment

Human keratinocyte HaCaT cells were grown at 37° C. under atmosphere with 95% humidity in the presence of 5% CO₂, in Dulbecco's modified Eagle's medium (DMEM, Gibco; Thermo Fisher Scientific, Inc., Waltham, Mass., USA) supplemented with 10% v/v fetal bovine serum (FBS, Gibco), 2 mM glutamine, 100 IU/ml penicillin, and 100 μg/mL of streptomycin (Gibco). HaCaT cells were dispensed into 6-well plates by 1×10⁶ cells/wells. After stabilization, 1 μM DEX and 0.1, 1, and 10 nM decursin were treated in the presence of 100 ng/mL of TNF-α for 24 hours.

6. Immunoblotting Assay

Proteins were extracted from the collected skin tissues and HaCaT cells using RIPA lysis buffer (Tech & Innovation, Gangwon, Korea) containing a protease inhibitor cocktail (Roche, Hoffmann, USA). Lysates were quantified by the Bradford method and denatured with dodecyl sulfate buffer at 98° C. for 5 minutes. Proteins were electrophoresed onto a polyvinylidene fluoride membrane. The membranes were incubated continuously with primary anti-caspase-3, -7, and -8, followed with secondary HRP-conjugated anti-rabbit and anti-mouse antibodies. Relative band densities were observed in ImageQuant TL (IQTL) software (GE Healthcare, IL, USA) and quantified using a computerized densitometry system (Image J, National Institutes of Health, Bethesda, Md., USA).

7. Statistical Analysis

Significance was determined by one-way analysis of variance (ANOVA) and Turkey's multiple comparison test. p<0.05 was taken to indicate statistical significance in all analyses.

8. Result of Experiment

1) Decursin Promoted the Growth of Hair Fibers.

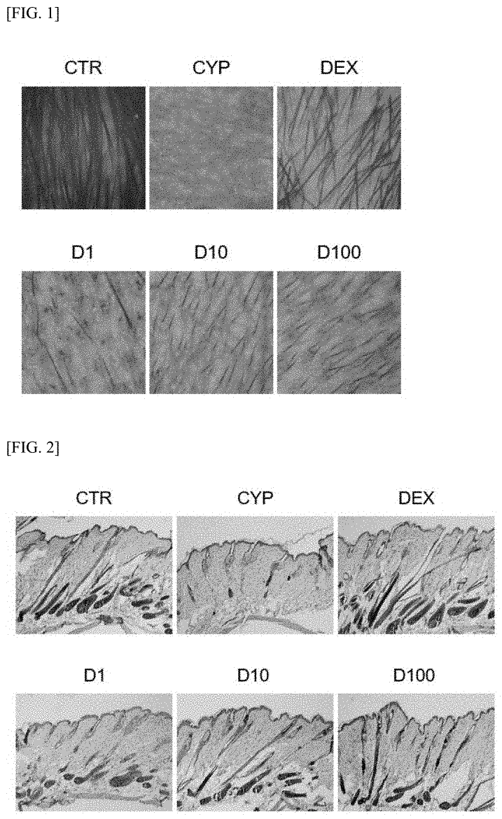

As shown in FIG. 1, it was observed that CYP injection caused a dystrophic change in hair growth in the bald skin using a digital dermatoscope, and new growth of hair fibers was induced through the epidermis by topical treatment of decursin. Moreover, compared to mice in the CYP group, hair growth was observed on the dorsal side in groups treated with DEX as well as all concentrations of decursin.

In addition, as shown in FIG. 2, the histological examination showed morphological changes in the hair follicles after decursin treatment. That is, compared to the untreated and uninjected CTR groups, it was estimated that the hair follicles were in the dystrophic catagen a week after the CYP injection, and the size of the bulge of the hair follicle was reduced by CYP. However, a number of hair follicles appeared in the subcutaneous layer after decursin treatment, indicating that the hair follicles were in the anagen. Compared to the CYP group, the shape of the ampulla of hair follicles was normalized in the decursin treated group while the hair shaft became straightened and appeared on the surface of the skin tissue.

2) Decursin Increased KGF Expression in Skin Tissues.

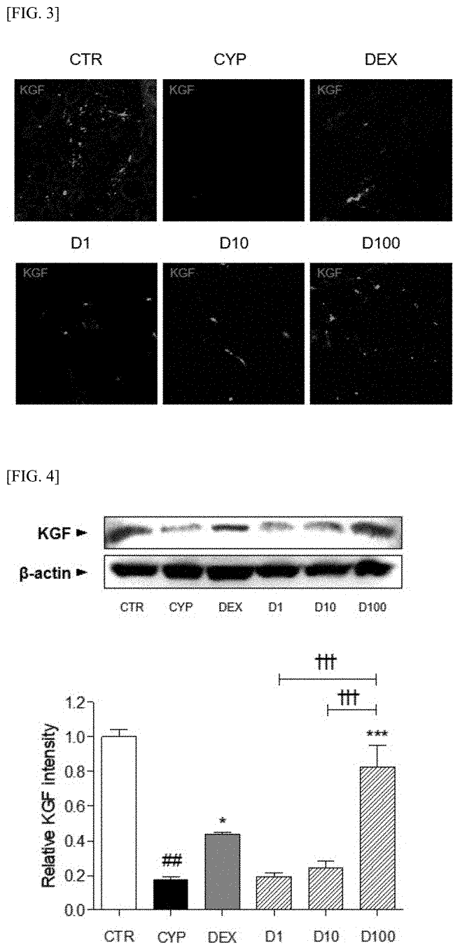

As shown in FIG. 3, compared to the skin tissue of the CTR group, CYP injection reduced KGF+ expression in the skin tissue, but KGF+ expression in the skin tissue was increased in CIA mice by treatment of 1, 10, and 100 μM decursin. As shown in FIG. 4, protein expression of KGF in CIA mice was significantly increased by treatment of 100 μM decursin.

3) Decursin Decreased Expression of Apoptosis Factors Including Caspase-3, -7, and -8 in Keratinocytes Stimulated with TNF-α.

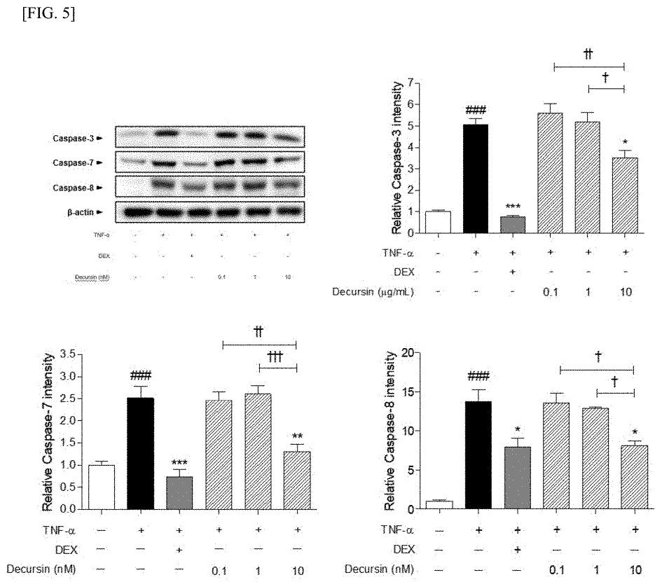

As shown in FIG. 5, caspase expression in TNF-α-treated HaCaT keratinocytes was significantly higher than in TNF-α-untreated cells, and the rates of increase for caspase-3, -7 and -8 in TNF-α-sensitive cells were 5.07, 2.52, and 13.70, respectively, compared to untreated cells. In addition, treatment of 10 nM decursin reduced, in a dose-dependent manner, an increase in the protein levels of caspase-3, -7, and -8 induced by TNF-α in HaCaT cells, and the rates of decrease for caspase-3, -7 and -8 were found to be 30.41%, 48.27%, and 41.07%, respectively.

Hereinafter, although formulation examples of the composition including decursin according to the present disclosure will be described, it is to only to describe the present disclosure in detail rather than limiting the present disclosure.

### <Formulation Example 1> Prescription Example of a Pharmaceutical Composition

### <Formulation Example 1-1> Preparation of Powder

20 mg of decursin, 100 mg of lactose, and 10 mg of talc were mixed and filled in an airtight bag to prepare powder.

### <Formulation Example 1-2> Preparation of Tablets

10 mg of decursin, 100 mg of corn starch, 100 mg of lactose, and 2 mg of magnesium stearate were mixed, and the mixture was tableted according to a conventional tablet preparation method to prepare tablets.

### <Formulation Example 1-3> Preparation of Capsules

10 mg of decursin, 100 mg of corn starch, 100 mg of lactose, and 2 mg of magnesium stearate were mixed. The ingredients above were mixed according to a conventional capsule preparation method and filled in a gelatin capsule to prepare a capsule.

### <Formulation Example 1-4> Preparation of an Injection

10 mg of decursin, an appropriate amount of sterile distilled water for injection, and an appropriate amount of a pH adjuster were mixed, and the preparation was performed with the above component content per 1 ampoule (2 ml) according to a conventional injection preparation method.

### <Formulation Example 2> Health Supplements

### <Formulation Example 2-1> Preparation of Health Food

1 mg of decursin, an appropriate amount of vitamin mixture (70 μg of vitamin A acetate, 1.0 mg of vitamin E, 0.13 mg of vitamin B1, 0.15 mg of vitamin B2, 0.5 mg of vitamin B6, 0.2 μg of vitamin B12, 10 mg of vitamin C, 10 μg of biotin, 1.7 mg of nicotinamide, 50 μg of folic acid, and 0.5 mg of calcium pantothenate) and an appropriate amount of an inorganic mixture (1.75 mg of ferrous sulfate, 0.82 mg of zinc oxide, 25.3 mg of magnesium carbonate, 15 mg of potassium phosphate monobasic, 55 mg of calcium phosphate dibasic, 90 mg of potassium citrate, 100 mg of calcium carbonate, and 24.8 mg of magnesium chloride) were mixed to prepare granules, and then health food was prepared according to a conventional method.

### <Formulation Example 2-2> Preparation of Health Drink

1 mg of decursin, 1000 mg of citric acid, 100 g of oligosaccharide, 2 g of plum concentrate, 1 g of taurine, and purified water were added to make a total volume of 900 ml.

After mixing the above ingredients according to a conventional health drink manufacturing method, stirring and heating were performed at 85° C. for about 1 hour. The prepared solution was filtrated and collected in a sterilized 2 L container to be stored in a fridge after being sealed and sterilized.

### <Formulation Example 3> Cosmetic Composition

### <Formulation Example 3-1> Preparation of a Hair Lotion

3.0 parts by weight of propylene glycol, 0.1 parts by weight of carboxypolymer, a trace amount of preservatives, and a remaining amount of purified water were heated to 80 to 85° C. while being stirred. Then, the mixture was put into a manufacturing unit and an emulsifier was operated. 1.0 parts by weight of polysorbate 60, 0.5 parts by weight of sorbitan sesquioleate, 10.0 parts by weight of liquid paraffin, 1.0 parts by weight of sorbitan stearate, 0.5 parts by weight of lyphophilic glycerin monostearate, 1.5 parts by weight of stearic acid, 1.0 parts by weight of glyceryl stearate/PEG-400 stearate, and 0.2 parts by weight of triethanolamine were heated to 80 to 85° C., followed by emulsification after being put. After the emulsification is completed, heat-cooling was performed to 50° C. while stirring using a stirrer, and a trace amount of fragrance was added. After cooling to 45° C., a trace amount of dye was added, and then decursin was added at 35° C., followed by aging after cooling to 25° C.

### <Prescription Example 2-2> Preparation of a Hair Cream

0.3 parts by weight of carboxypolymer, 5.0 parts by weight of butylene glycol, 3.0 parts by weight of glycerin, and the remaining amount of purified water were heated to 80 to 85° C. while being stirred. Then, the mixture was put into the manufacturing unit, and the emulsifier was operated. 2.0 parts by weight of stearic acid, 2.0 parts by weight of cetyl alcohol, 2.0 parts by weight of glyceryl monostearate, 0.5 parts by weight of polyoxyethylene sorbitan monostearate, 0.5 parts by weight of sorbitan sesquioleate, 1.0 parts by weight of glyceryl monostearate/glyceryl stearate/polyoxyethylene stearate, 1.0 parts by weight of wax, 4.0 parts by weight of liquid paraffin, 4.0 parts by weight of squalane, and 4.0 parts by weight of caprylic/capric triglyceride were heated to 80 to 85° C. and then put, followed by emulsification by adding 0.5 parts by weight of triethanolamine. After the emulsification was completed, cooling was performed to 35° C. while stirring with a stirrer, then decursin was added, followed by aging after cooling to 25° C.

### <Prescription Example 2-3> Preparation of a Hair Shampoo

1 part by weight of decursin, 1.5 parts by weight of silicone oil, 1.0 parts by weight of silicone gum, 12.0 parts by weight of ammonium lauryl sulfate, 4.0 parts by weight of polyoxyethylene lauryl ammonium sulfate, 3.0 parts by weight of cocamide DEA, 1.0 parts by weight of glycol stearate, 3.0 parts by weight of lauramidopropyl betaine, 3.0 parts by weight of lauroamphocarboxyglycinate, and appropriate amounts of color, fragrance and preservative were added to prepare a hair shampoo.

As the specific parts of the present disclosure have been described in detail above, for those of ordinary skill in the art, it is clear that these specific descriptions are only preferred example embodiments, and the scope of the present disclosure is not limited thereby. Accordingly, the substantial scope of the present disclosure will be defined by the appended claims and their equivalents.

What is claimed is:

1. A pharmaceutical composition for treating or preventing alopecia, the pharmaceutical composition comprising decursin or a pharmaceutically acceptable salt thereof.

2. The pharmaceutical composition of claim 1, wherein the decursin is extracted from a root of Angelica gigas.

3. The pharmaceutical composition of claim 1, wherein the decursin promotes expression of keratinocyte growth factor (KGF) in skin tissues, prevents apoptosis of hair follicle cells, and promotes hair growth.

4. The pharmaceutical composition of claim 1, wherein the alopecia is one or more selected from the group consisting of androgenetic alopecia, telogen alopecia, drug-induced alopecia, mechanical alopecia, traumatic alopecia, pressure-induced alopecia, anagen alopecia, dandruff-induced alopecia, syphilitic alopecia, seborrheic alopecia, symptomatic alopecia, scarring alopecia, congenital alopecia, alopecia areata, tinea capitis, frontal alopecia, hypotrichosis, hereditary hypotrichosis simplex, and alopecia universalis.

5. The pharmaceutical composition of claim 1, wherein the composition is form of an external preparation.

6. The pharmaceutical composition of claim 5, wherein the external preparation is provided in a formulation selected from the group consisting of ointments, creams, gels, patches, sprays, plasters, lotions, liniment agents, pastas, and cataplasmas.

7. A health functional food composition for preventing or ameliorating alopecia comprising decursin.

8. A cosmetic composition for preventing or ameliorating alopecia comprising decursin.

9. The cosmetic composition of claim 8, wherein the composition is provided in a formulation selected from the group consisting of hair tonic, hair conditioner, hair essence, hair lotion, hair nutrition lotion, hair shampoo, hair conditioner, hair treatment, hair cream, hair nourishment cream, hair moisture cream, hair massage cream, hair wax, hair aerosol, hair pack, hair nourishment pack, hair soap, hair cleansing foam, hair oil, hair dryer, hair preservative, hair dye, hair wave agent, hair bleach, hair gel, hair glaze, hair dresser, hair lacquer, hair moisturizer, hair mousse, and hair spray.
